(19) United States
(12) Patent Application Publication
(10) Pub. No.: US 2015/0025279 A1
(43) Pub. Date: Jan. 22, 2015

(54) PROCESS FOR PRODUCING DODECANE-1,12-DIOL BY REDUCTION OF LAURYL LACTONE PRODUCED FROM THE OXIDATION OF CYCLODODECANONE

(71) Applicant: INVISTA North America S.a.r.l., Wilmington, DE (US)

(72) Inventors: James D. Hastings, Victoria, TX (US); Frank E. Herkes, Wilmington, DE (US); Gurusamy Rajendran, League City, TX (US); Qun Sun, Wilmington, DE (US)

(73) Assignee: INVISTA North America S.a.r.l., Wilmington, DE (US)

(21) Appl. No.: 14/375,739

(22) PCT Filed: Jan. 21, 2013
(86) PCT No.: PCT/US2013/022408

§ 371 Date: Jul. 30, 2014

Related U.S. Application Data
(60) Provisional application No. 61/593,452, filed on Feb. 1, 2012.

(51) Int. Cl. C07C 29/147; C07D 313/00

(52) U.S. Cl. CPC C07C 29/147; C07D 313/00
USPC 568/864

(57) ABSTRACT

A process for synthesizing dodecane-1,12-diol, and by-products thereof, by the reduction of lauryl lactone produced from the oxidation of cyclododecanone.

### CROSS REFERENCE TO RELATED APPLICATION

This application claims benefit of priority from Provisional Application No. 61/593,452, filed Feb. 1, 2012. This application hereby incorporates by reference Provisional Application No. 61/593,452 in its entirety.

### FIELD OF THE INVENTION

The invention relates to a method for the synthesis of dodecane-1,12-diol.

### BACKGROUND OF THE INVENTION

Dodecane-1,12-diol is a high-purity, 12-carbon, linear diol. It is a highly versatile monomer and chemical intermediate for use in applications where excellent hydrolytic, oxidative, and thermal stability are desired. Other applications of dodecane-1,12-diol include, e.g., fragrances, synthetic lubricants, elastomers, adhesives, polymer crosslinkers, pharmaceuticals, polyesters and co-polyesters, detergents, inks, and polyester polyols for polyurethanes.

Known preparations of dodecane-1,12-diol include the conversion of 1,12-dodecanedioic acid (DDDA) to the methyl ester (dimethyl 1,12-dodecanedioate, DMDD) by a non-catalytic esterification process. After purification of the ester it undergoes hydrogenation to dodecane-1,12-diol.

### SUMMARY OF THE INVENTION

The invention provides for a method of producing dodecane-1,12-diol that includes oxidizing cyclododecanone to provide lauryl lactone, and reducing the lauryl lactone to provide dodecane-1,12-diol. The crude oxidation product, lauryl lactone, can include the by-product maleic acid, which can precipitate from the reaction product, thereby advantageously enhancing the conversion and/or rate of cyclododecanone to provide lauryl lactone. Use of a low-boiling point organic solvent can control the exothermic heat by solvent reflux at the reaction temperature. The crude oxidation product, lauryl lactone (which can include by-products, as well as un-reacted reagents and un-reacted starting material) can subsequently be directly hydrogenated to provide dodecane-1,12-diol. The hydrogenation can optionally be carried out in the presence of water, organic solvent and catalyst. The dodecane-1,12-diol is thereby obtained in relatively high yield and selectivity, without the need for an intermediate work-up. Additionally, both reactions can be carried out on a commercial (e.g., kilogram or multi-kilogram) scale.

The invention provides for a method of producing dodecane-1,12-diol. The method includes oxidizing cyclododecanone to provide lauryl lactone, and reducing the lauryl lactone to the dodecane-1,12-diol. Additional by-products can be obtained in the oxidation that include, e.g., maleic acid. As such, the invention also provides for optionally converting, via the reducing (i.e., the reduction), maleic acid to butane-1,4-diol.

The invention also provides for a method of producing additional dodecane-1,12-diol. The method includes oxidizing cyclododecanone to provide lauryl lactone, and the by-products maleic acid, 1,12-dodecanedioic acid and/or the 12-hydroxydodecanoic acid. Upon subjecting the crude lauryl lactone product to the reduction (e.g., hydrogenation), the by-products 1,12-dodecanedioic acid and/or 12-hydroxydodecanoic acid are converted to dodecane-1,12-diol.

The invention also provides for a method of producing butane-1,4-diol. The method includes oxidizing cyclododecanone to provide lauryl lactone. The crude lauryl lactone includes the by-product maleic acid. Upon subjecting the crude lauryl lactone product to the reduction (e.g., hydrogenation), the by-product maleic acid is reduced to butane-1,4-diol.

### BRIEF DESCRIPTION OF THE FIGURES

FIG. 1 illustrates a process for synthesizing dodecane-1,12-diol, and by-products thereof, by the reduction of lauryl lactone produced from the oxidation of cyclododecanone.

### DETAILED DESCRIPTION OF THE INVENTION

Reference will now be made in detail to certain claims of the disclosed subject matter. While the disclosed subject matter will be described in conjunction with the enumerated claims, it will be understood that they are not intended to limit the disclosed subject matter to those claims. On the contrary, the disclosed subject matter is intended to cover all alternatives, modifications, and equivalents, which can be included within the scope of the presently disclosed subject matter as defined by the claims.

References in the specification to “one embodiment,” “an embodiment,” “an example embodiment,” etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

In this document, the terms “a” or “an” are used to include one or more than one and the term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading can occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods of manufacturing described herein, the steps can be carried out in any order without departing from the principles of the invention, except when a temporal or operational sequence is explicitly recited. Recitation in a claim to the effect that first a step is performed, then several other steps are subsequently performed, shall be taken to mean that the first step is performed before any of the other steps, but the other steps can be performed in any suitable sequence, unless a sequence is further recited within the other steps. For example, claim elements that recite “Step A, Step B, Step C, Step D, and Step E” shall be construed to mean step A is carried out first, step E is carried out last, and steps B, C, and D can be carried out in any sequence between steps A and E, and that the sequence still falls within the literal scope of the claimed process. A given step or sub-set of steps can also be repeated, or carried out simultaneously with other steps. In another example, claim elements that recite “Step A, Step B, Step C, Step D, and Step E” can be construed to mean Step A is carried out first, Step B is carried out next, Step C is carried out next, Step D is carried out next, and Step E is carried out last.

Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

### DEFINITIONS

Unless stated otherwise, the following terms and phrases as used herein are intended to have the following meanings:

The singular forms “a,” “an” and “the” can include plural referents unless the context clearly dictates otherwise.

The term “about” can allow for a degree of variability in a value or range, for example, within 10%, within 5%, or within 1% of a stated value or of a stated limit of a range. When a range or a list of sequential values is given, unless otherwise specified any value within the range or any value between the given sequential values is also disclosed.

The term “dodecane-1,12-diol,” “C12 LD,” “C12LD,” “C12 linear diol,” “1,12-dodecanediol” “dodecamethylene glycol,” or “HO(CH₂)₁₂OH” refers to a compound having the CAS Reg. No. [5675-51-4], the molecular formula C₁₂H₂₆O₂, and the structural formula:

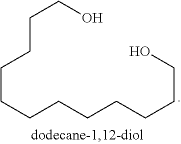 [SMILES: OCCCCCCCCCCCCO]

The term “cyclododecanone” or “CDDK” refers to a compound of the structural formula:

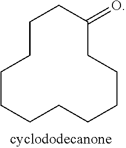 [SMILES: O=C1CCCCCCCCCCC1]

The term “lauryl lactone” or “LLON” refers to a compound of the structural formula:

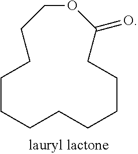 [SMILES: O=C1CCCCCCCCCCCO1]

The term “butane-1,4-diol” or “BDO” refers to a compound of the structural formula:

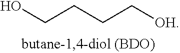

The term “maleic anhydride” or “MAN” refers to a compound of the structural formula:

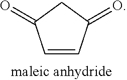

The term “maleic acid” or “MA” refers to a compound of the structural formula;

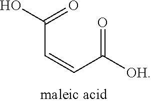

The term “12-hydroxydodecanoic acid” or “hydroxydodecanoic acid” refers to a compound of the structural formula:

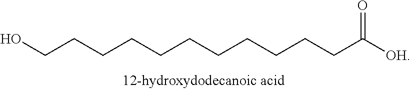 [SMILES: O=C(O)CCCCCCCCCCCO]

The term “1,12-dodecanedioic acid” or “dodecanedioic acid” refers to a compound of the structural formula:

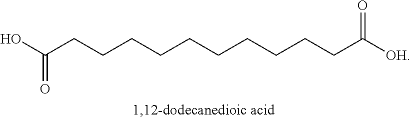 [SMILES: O=C(O)CCCCCCCCCCC(=O)O]

The term “methyl 12-hydroxydodecanoate,” “12-hydroxydodecanoate,” “methyl 12-hydroxydodecanoate,” “methyl 12-hydroxydodecanoic acid,” or “methyl ester of 12-hydroxydodecanoic acid” refers to a compound of the structural formula:

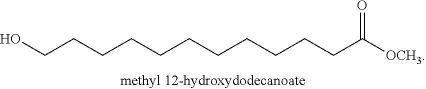 [SMILES: COC(=O)CCCCCCCCCCCO]

The term “1-dodecanol” refers to a compound of the structural formula:

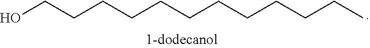

The term “12-methoxy-12-oxododecanoic acid” or “mono methyl ester of 1,12-dodecadioic acid” refers to a compound of the structural formula:

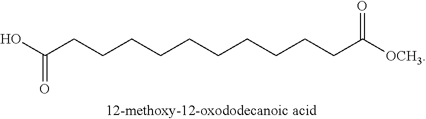 [SMILES: COC(=O)CCCCCCCCCCC(=O)O]

The term “1-undecanol” refers to a compound of the structural formula:

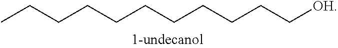

The term hydrogen peroxide refers to the compound H₂O₂. For use in the methods of the invention, the hydrogen peroxide will typically be about 70% H₂O₂ in water, and is commercially available from, e.g., Arkema (Philadelphia, Pa.).

It is appreciated that those of skill in synthetic organic chemistry understand that reagents are typically referred to by the chemical names that they bear or formulae that represent their structures prior to addition to a chemical reaction mixture, even though the chemical species actually present in the reaction mixture or involved in the reaction may be otherwise. While a compound may undergo conversion to a compound bearing a different name or represented by a different formula prior to or during a specified reaction step, reference to these compounds by their original name or formula is acceptable and is well-understood by those of skill in the art of organic chemistry.

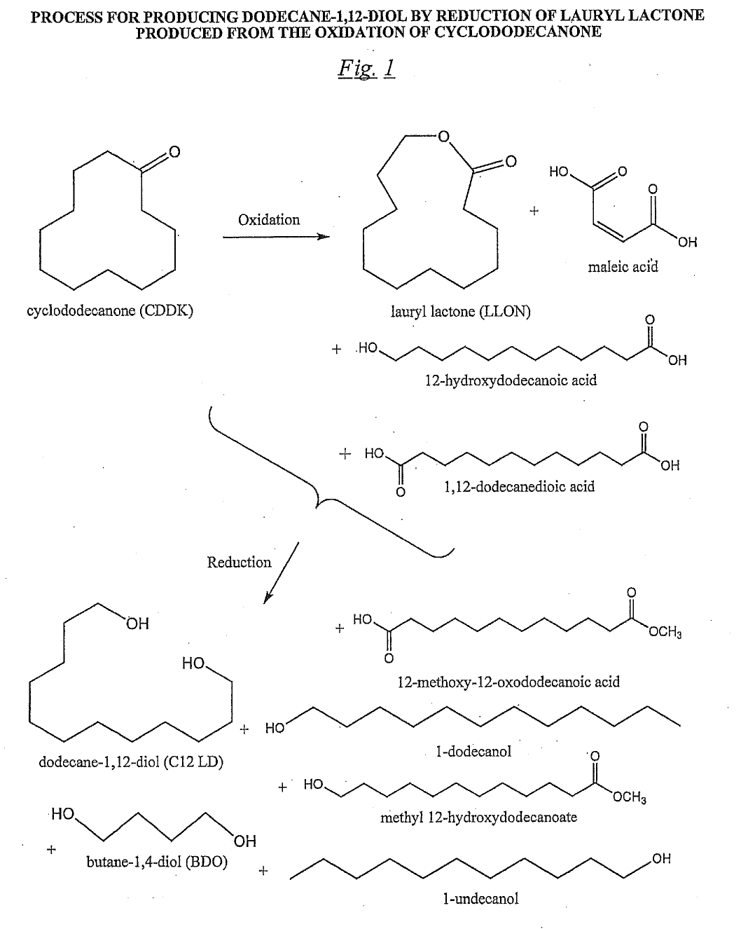

Referring to FIG. 1, a process for synthesizing dodecane-1,12-diol is provided. The synthesis includes the oxidation of cyclododecanone to provide crude lauryl lactone (e.g., lauryl lactone and optional by-products, un-reacted starting material, solvent and/or reagents). The oxidation of cyclododecanone to lauryl lactone can be carried out, e.g., via a “Baeyer-Villiger” oxidation. The optional by-products obtained via the Baeyer-Villiger oxidation of cyclododecanone can include, e.g., maleic acid, and/or 12-hydroxydodecanoic acid, and/or 1,12-dodecanedioic acid.

The crude oxidation product is then reduced, to provide crude dodecane-1,12-diol (e.g., dodecane-1,12-diol and optional by-products, un-reacted starting material, solvent and/or reagents). The reduction can be carried out via a “hydrogenation” reduction. The optional by-products obtained via the hydrogenation of crude lauryl lactone can include, e.g., butane-1,4-diol, 12-methoxy-12-oxododecanoic acid, 1-dodecanol, methyl 12-hydroxydodecanoate, and/or 1-undecanol.

### Oxidation

Various embodiments of the invention provide for a method of producing dodecane-1,12-diol, wherein the method includes oxidizing cyclododecanone to provide lauryl lactone.

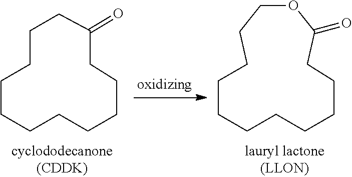 [SMILES: O=C1CCCCCCCCCCC1 ; O=C1CCCCCCCCCCCO1]

Various embodiments of the invention also provide for a method of producing dodecane-1,12-diol, wherein the method includes oxidizing cyclododecanone to provide lauryl lactone and the by-product maleic acid.

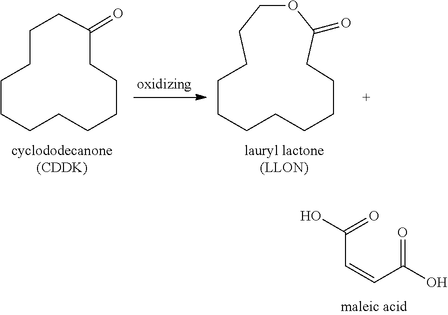 [SMILES: O=C1CCCCCCCCCCC1 ; O=C1CCCCCCCCCCCO1 ; O=C(O)/C=C\C(=O)O]

Various embodiments of the invention also provide for a method of producing dodecane-1,12-diol, wherein the method includes oxidizing cyclododecanone to provide lauryl lactone and the by-products maleic acid, 12-hydroxydodecanoic acid and/or 1,12-dodecanedioic acid.

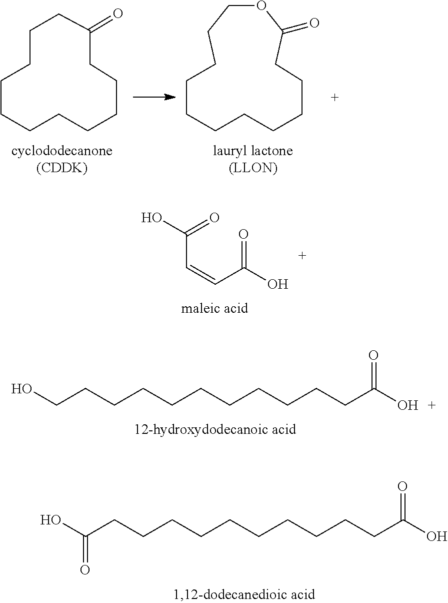 [SMILES: O=C1CCCCCCCCCCC1 ; O=C1CCCCCCCCCCCO1 ; O=C(O)/C=C\C(=O)O ; O=C(O)CCCCCCCCCCCO ; O=C(O)CCCCCCCCCCC(=O)O]

The oxidation described herein is carried out to effectively provide the lauryl lactone in a suitable yield, purity and/or selectivity. The reagents, solvents and/or reaction conditions are selected, such that the subsequent reduction can be carried out without any need for an intermediate work-up.

In specific embodiments of the invention, the oxidation is carried out via a “Baeyer-Villiger” or “BV” oxidation that employs peroxy acids or hydrogen peroxide. Suitable “Baeyer-Villiger” reagents and reaction conditions are disclosed, e.g., in Compendium of Organic Synthetic Methods (John Wiley & Sons, New York) Vol. 1, Ian T. Harrison and Shuyen Harrison (1971); Vol. 2, Ian T. Harrison and Shuyen Harrison (1974); Vol. 3, Louis S. Hegedus and Leroy Wade (1977); Vol. 4, Leroy G. Wade Jr., (1980); Vol. 5, Leroy G. Wade Jr. (1984); and Vol. 6, Michael B. Smith; as well as March, J., Advanced Organic Chemistry, 3rd Edition, John Wiley & Sons, New York (1985); Comprehensive Organic Synthesis. Selectivity, Strategy & Efficiency in Modern Organic Chemistry, In 9 Volumes, Barry M. Trost, Editor-in-Chief, Pergamon Press, New York (1993); Advanced Organic Chemistry, Part B: Reactions and Synthesis, 4th Ed.; Carey and Sundberg; Kluwer Academic/Plenum Publishers: New York (2001); Advanced Organic Chemistry, Reactions, Mechanisms, and Structure, 2nd Edition, March, McGraw Hill (1977); and Comprehensive Organic Transformations, 2nd Edition, Larock, R. C., John Wiley & Sons, New York (1999).

Exemplary reagents useful in the oxidation include, e.g., hydrogen peroxide (H₂O₂), meta-chloroperoxybenzoic acid (mCPBA), trifluoro peracetic acid (CF₃CO₃H), performic acid (CH₂O₃), permaleic acid (PMA), peracetic acid (CH₃CO₃H), magnesium monoperoxyphthalate (MMPP), perbenzoic acid (PBA) and monoperphthalic acid (MPPA).

In specific embodiments of the invention, the oxidation employs the reagents hydrogen peroxide and maleic anhydride, and the non-reactive solvent methyl acetate.

In specific embodiments of the invention, the oxidation employs the reagents hydrogen peroxide and maleic anhydride, and the non-reactive solvent methyl acetate. In further specific embodiments, the reaction is carried out by the simultaneous addition of the hydrogen peroxide and the maleic anhydride in the methyl acetate, to a solution of the cyclododecanone in the methyl acetate.

In specific embodiments of the invention, the oxidation employs the reagents hydrogen peroxide and maleic anhydride. In further specific embodiments, the reaction is carried out by the addition of the cyclododecanone to a preformed solution of hydrogen peroxide and maleic anhydride.

In specific embodiments of the invention, the oxidation employs the reagents hydrogen peroxide and maleic anhydride, and the non-reactive solvent methyl acetate. In further specific embodiments, the reaction is carried out by the addition of hydrogen peroxide to a solution of cyclododecanone and maleic anhydride, in the methyl acetate.

In specific embodiments of the invention, the oxidation employs the solvent dioxane, ethyl propionate, ethyl acetate, dibasic esters, 1,12-dimethyldodecanediol, methyl acetate, ω-pentadecalactone, or a combination thereof. In more specific embodiments of the invention, the oxidation employs the solvent methyl acetate.

The oxidation described herein will typically be carried out employing starting material (i.e., cyclododecanone) and reagents (e.g., hydrogen peroxide and maleic anhydride), in specified and predetermined molar amounts and ratios. For example, each of the reagents employed in the oxidation can independently be employed in a molar excess, each relative to the cyclododecanone. Such amounts and ratios are typically selected in an attempt to drive the reaction to completion, thereby increasing the yield of desired product.

In specific embodiments of the invention, the oxidation employs the reagent hydrogen peroxide, present in about 1.5 molar equivalents to about 2.5 molar equivalents, relative to the cyclododecanone. In more specific embodiments of the invention, the oxidation employs the reagent hydrogen peroxide, present in about 1.7 molar equivalents to about 2.3 molar equivalents, relative to the cyclododecanone. In more specific embodiments of the invention, the oxidation employs the reagent hydrogen peroxide, present in about 1.9 molar equivalents to about 2.1 molar equivalents, relative to the cyclododecanone.

In specific embodiments of the invention, the oxidation employs the reagents maleic anhydride and hydrogen peroxide, present in a molar ratio of about 0.5 to about 1.5. In more specific embodiments of the invention, the oxidation employs the reagents maleic anhydride and hydrogen peroxide, present in a molar ratio of about 0.7 to about 1.3. In more specific embodiments of the invention, the oxidation employs the reagents maleic anhydride and hydrogen peroxide, present in a molar ratio of about 0.85 to about 1.0.

In specific embodiments of the invention, the oxidation is carried out employing a solvent having a boiling point less than about 80° C. In more specific embodiments of the invention, the oxidation is carried out employing a solvent having a boiling point less than about 70° C. In more specific embodiments of the invention, the oxidation is carried out employing a solvent having a boiling point less than about 60° C. Relatively low boiling solvents can be used to control the exothermic heat by solvent reflux at the desired reaction temperature.

In specific embodiments of the invention, the oxidation is carried out at a temperature of about 45° C. to about 60° C.

In specific embodiments of the invention, the oxidation is carried out for a period of time of about 12 hours to about 36 hours. In more specific embodiments of the invention, the oxidation is carried out for a period of time of about 15 hours to about 30 hours. In more specific embodiments of the invention, the oxidation is carried out for a period of time of about 18 hours to about 24 hours.

In specific embodiments of the invention, the oxidation provides lauryl lactone in at least about a 92 mol.% yield, relative to the cyclododecanone. In more specific embodiments of the invention, the oxidation provides lauryl lactone in at least about a 95 mol.% yield, relative to the cyclododecanone. In more specific embodiments of the invention, the oxidation provides lauryl lactone in at least about a 98 mol.% yield, relative to the cyclododecanone. In more specific embodiments of the invention, the oxidation provides lauryl lactone in at least about a 99 mol.% yield, relative to the cyclododecanone.

In specific embodiments of the invention, the oxidation provides lauryl lactone in at least about a 75 mol.% selectivity, relative to the cyclododecanone. In more specific embodiments of the invention, the oxidation provides lauryl lactone in at least about a 80 mol.% selectivity, relative to the cyclododecanone. In more specific embodiments of the invention, the oxidation provides lauryl lactone in about 80 mol.% selectivity to about 90 mol.% selectivity, relative to the cyclododecanone.

In specific embodiments of the invention, the oxidation is carried out in a batch mode, wherein at least about 20 kg of lauryl lactone is obtained, per batch. In more specific embodiments of the invention, the oxidation is carried out in a batch mode, wherein at least about 50 kg of lauryl lactone is obtained, per batch. In more specific embodiments of the invention, the oxidation is carried out in a batch mode, wherein at least about 100 kg of lauryl lactone is obtained, per batch. In more specific embodiments of the invention, the oxidation is carried out in a batch mode, wherein at least about 500 kg of lauryl lactone is obtained, per batch. In more specific embodiments of the invention, the oxidation is carried out in a batch mode, wherein at least about 1,000 kg of lauryl lactone is obtained, per batch.

In specific embodiments of the invention, maleic acid is produced as a by-product in the oxidizing, and is allowed to precipitate from the reaction mixture. Precipitation of the by-product maleic acid can enhance the conversion and/or rate of cyclododecanone to lauryl lactone.

In specific embodiments of the invention, the oxidizing produces one or more by-products comprising at least one of 12-hydroxdodecanoic acid, maleic acid, 1,12-dodecanedioic acid, and un-reacted starting material. In more specific embodiments of the invention, the oxidizing produces the by-products 12-hydroxdodecanoic acid, maleic acid, and 1,12-dodecanedioic acid.

### Reduction

Various embodiments of the invention provide for a method of producing dodecane-1,12-diol that includes reducing lauryl lactone, obtained from the oxidization of the cyclododecanone to the lauryl lactone, to provide dodecane-1,12-diol.

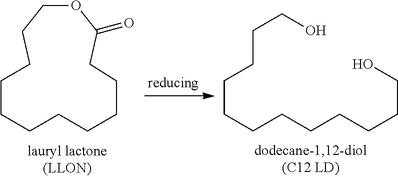 [SMILES: O=C1CCCCCCCCCCCO1 ; OCCCCCCCCCCCCO]

Various embodiments of the invention provide for a method of producing dodecane-1,12-diol that includes reducing lauryl lactone, obtained from the oxidization of the cyclododecanone to the lauryl lactone, to provide dodecane-1,12-diol and the by-product butane-1,4-diol.

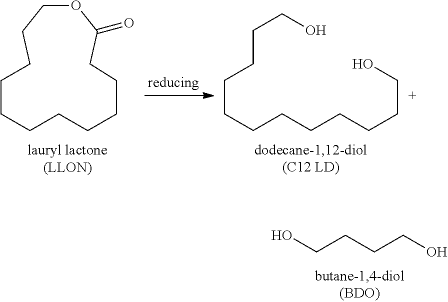 [SMILES: O=C1CCCCCCCCCCCO1 ; OCCCCCCCCCCCCO ; OCCCCO]

Various embodiments of the invention provide for a method of producing dodecane-1,12-diol that includes reducing lauryl lactone, obtained from the oxidization of the cyclododecanone to the lauryl lactone, to provide dodecane-1,12-diol and the by-products butane-1,4-diol, 12-methoxy-12-oxododecanoic acid, 1-dodecanol, methyl 12-hydroxydodecanoate, and/or 1-undecanol.

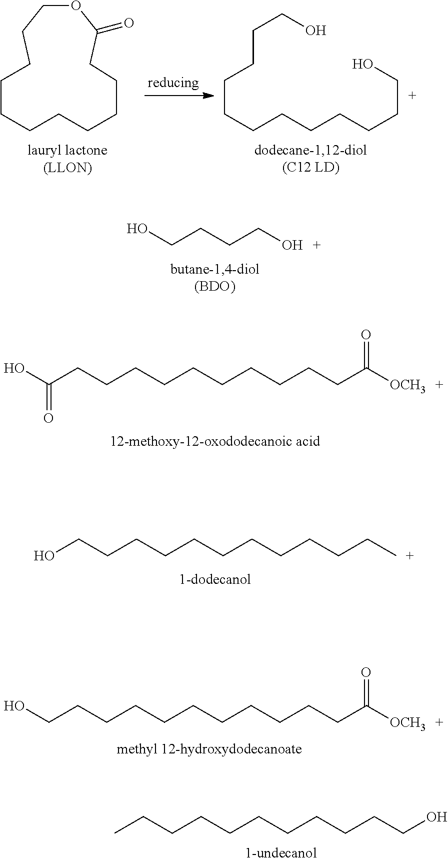

Various embodiments of the invention provide for a method of producing dodecane-1,12-diol that includes reducing 12-hydroxydodecanoic acid and/or 1,12-dodecanedioic acid, the by-products from the oxidization of the cyclododecanone to the lauryl lactone, to provide dodecane-1,12-diol.

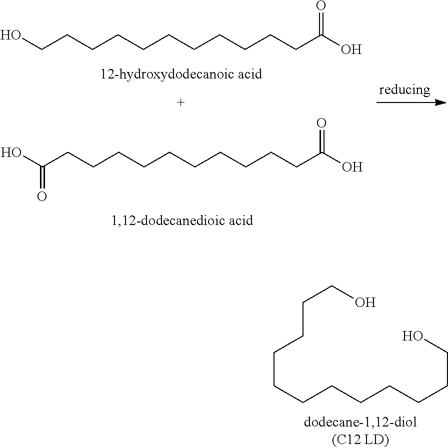 [SMILES: O=C(O)CCCCCCCCCCCO ; O=C(O)CCCCCCCCCCC(=O)O ; OCCCCCCCCCCCCO]

Various embodiments of the invention provide for a method of co-producing butane-1,4-diol that includes reducing maleic acid, a by-product from the oxidization of the cyclododecanone to the lauryl lactone, to butane-1,4-diol.

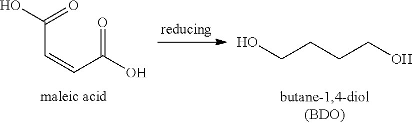 [SMILES: O=C(O)/C=C\C(=O)O ; OCCCCO]

The reduction described herein is carried out to effectively provide the dodecane-1,12-diol in a suitable yield, purity and/or selectivity. The reagents, catalysts, solvents and/or reaction conditions are selected, such that the reduction can be carried out without any need for an intermediate work-up from the preceding oxidation. The reduction (e.g., hydrogenation) can be carried out on the crude lauryl lactone without any need for an intermediate work-up. As such, the reduction (e.g., hydrogenation) can be carried out on the crude lauryl lactone in the presence of, e.g., un-reacted starting material from the oxidation (i.e., cyclododecanone), un-reacted reagent from the oxidation (e.g., hydrogen peroxide and maleic anhydride), solvent from the oxidation (e.g., methyl acetate), and any by-products obtained in the oxidation (e.g., maleic acid, 12-hydroxydodecanoic acid, and/or 1,12-dodecanedioic acid).

Because the reduction (e.g., hydrogenation) can be carried out without any need for an intermediate work-up from the oxidation, by-products from the oxidation can be subject to the reagents, solvent and reaction conditions of the reduction. Specifically, when the oxidation is a Baeyer-Villiger oxidation, the by-products obtained can include maleic acid, 12-hydroxydodecanoic acid, and 1,12-dodecanedioic acid. Upon being subject to the reduction (e.g. hydrogenation) reagents, solvent and reaction conditions the maleic acid can be reduced to butane-1,4-diol. As such, the process of the invention can provide the commercially valuable co-product butane-1,4-diol, which has a commercial use. Additionally, upon being subject to the reduction (e.g. hydrogenation) reagents, solvent and reaction conditions the 12-hydroxydodecanoic acid, and/or 1,12-dodecanedioic acid can be reduced to dodecane-1,12-diol. This will effectively provide for an additional amount of desired product, thereby increasing the yield of dodecane-1,12-diol.

In specific embodiments of the invention, the reduction is carried out via a “hydrogenation” that employs hydrogen gas (H₂), catalyst and solvent. Suitable “hydrogenation” reagents, catalysts, solvents, and reaction conditions are disclosed, e.g., in Compendium of Organic Synthetic Methods (John Wiley & Sons, New York) Vol. 1, Ian T. Harrison and Shuyen Harrison (1971); Vol. 2, Ian T. Harrison and Shuyen Harrison (1974); Vol. 3, Louis S. Hegedus and Leroy Wade (1977); Vol. 4, Leroy G. Wade Jr., (1980); Vol. 5, Leroy G. Wade Jr. (1984); and Vol. 6, Michael B. Smith; as well as March, J., Advanced Organic Chemistry, 3rd Edition, John Wiley & Sons, New York (1985); Comprehensive Organic Synthesis. Selectivity, Strategy & Efficiency in Modem Organic Chemistry, In 9 Volumes, Barry M. Trost, Editor-in-Chief, Pergamon Press, New York (1993); Advanced Organic Chemistry, Part B: Reactions and Synthesis, 4th Ed.; Carey and Sundberg; Kluwer Academic/Plenum Publishers: New York (2001); Advanced Organic Chemistry, Reactions, Mechanisms, and Structure, 2nd Edition, March, McGraw Hill (1977); and Comprehensive Organic Transformations, 2nd Edition, Larock, R. C., John Wiley & Sons, New York (1999).

With rare exceptions, no reaction below about 480° C. (750 K or 900° F.) occurs between H₂ and organic compounds in the absence of metal catalysts. The catalyst binds both the H₂ and the unsaturated substrate and facilitates their union. Platinum, palladium, rhodium, and ruthenium form highly active catalysts, which operate at lower temperatures and lower pressures of H₂. Non-precious metal catalysts, especially those based on nickel (such as Raney nickel and Urushibara nickel) have also been developed as economical alternatives, but they are often slower or require higher temperatures. The trade-off is activity (speed of reaction) vs. cost of the catalyst, and cost of the apparatus required for use of high pressures.

Two broad families of catalysts are known, homogeneous catalysts and heterogeneous catalysts. Homogeneous catalysts dissolve in the solvent that contains the unsaturated substrate. Heterogeneous catalysts are solids that are suspended in the same solvent with the substrate or are treated with gaseous substrate.

Exemplary homogeneous catalysts include the rhodium-based compound known as “Wilkinson's” catalyst and the iridium-based “Crabtree's” catalyst.

Heterogeneous catalysts for hydrogenation are more common industrially. As in homogeneous catalysts, the activity is adjusted through changes in the environment around the metal, i.e., the coordination sphere. Different faces of a crystalline heterogeneous catalyst display distinct activities, for example. Similarly, heterogeneous catalysts are affected by their supports, i.e., the material upon with the heterogeneous catalyst is bound.

In specific embodiments of the invention, the reducing is a hydrogenation.

In specific embodiments of the invention, the reducing employs a homogeneous catalyst. In other specific embodiments of the invention, the reducing employs a heterogeneous catalyst.

In specific embodiments of the invention, the reducing employs hydrogen gas (H₂), solvent and a catalyst, at an elevated pressure and an elevated temperature. In more specific embodiments of the invention, the reducing employs hydrogen gas (H₂), catalyst, (C₁-C₆)alkyl substituted with hydroxyl, and water. In more specific embodiments of the invention, the reducing employs hydrogen gas (H₂), catalyst, methanol, and water.

Any suitable solvent can be employed in the hydrogenation, provided the lauryl lactone is effectively reduced to dodecane-1,12-diol in a suitable yield, purity and/or selectivity. The solvent can also be selected, e.g., such that the catalyst and reagent (e.g., hydrogen gas) can retain their effectiveness. The solvent can also be selected, such that by-products of the oxidation can be subject to the hydrogenation, without significantly producing any undesired by-products in the hydrogenation. Suitable solvents include, e.g., low molecular weight alcohols, such as (C₁-C₆)alkyl substituted with hydroxyl. Specific suitable solvents that can be employed in the hydrogenation include, e.g., methanol and ethanol. Because of the propensity of 12-hydroxydodecanoic and 1,12-dodceanedioic acids to form C24 dimer esters during the reduction, methanol can be employed in the reduction to convert these acids and lactone to methyl esters, which are easily reduced under the reaction conditions.

In specific embodiments of the invention, the reducing employs ruthenium (Ru) on carbon catalyst. In more specific embodiments of the invention, the reducing employs 2% ruthenium (Ru) on carbon catalyst. In more specific embodiments of the invention, the reducing employs 2% ruthenium (Ru) on carbon catalyst containing Re, Sn or a combination thereof. In more specific embodiments of the invention, the reducing employs 2% ruthenium (Ru) on carbon catalyst containing Re and Sn.

The addition of rhenium to the Ru catalyst can enhance the activity of the catalyst. Sn addition can decrease hydrogenolysis during the reduction process. Additionally, reaction water (i.e., water present from the hydrogen peroxide) can be supplemented during the reduction (e.g., hydrogenation) with additional water, to enhance the catalyst effectiveness.

In specific embodiments of the invention, the reducing is carried out at an elevated pressure of at least about 500 psig. In more specific embodiments of the invention, the reducing is carried out at an elevated pressure of at least about 1000 psig. In more specific embodiments of the invention, the reducing is carried out at an elevated pressure of at least about 2000 psig. In more specific embodiments of the invention, the reducing is carried out at an elevated pressure of about 2000 psig to about 3000 psig.

In specific embodiments of the invention, the reducing is carried out at an elevated temperature of at least about 100° C. In more specific embodiments of the invention, the reducing is carried out at an elevated temperature of at least about 150° C. In more specific embodiments of the invention, the reducing is carried out at an elevated temperature of at least about 170° C. hi more specific embodiments of the invention, the reducing is carried out at an elevated temperature of about 180° C. to about 250° C.

In specific embodiments of the invention, the reducing is carried out at for at least about 12 hours. In more specific embodiments of the invention, the reducing is carried out at for at least about 18 hours. In more specific embodiments of the invention, the reducing is carried out at for at least about 22 hours. In more specific embodiments of the invention, the reducing is carried out at for about 22 hours to about 28 hours.

In specific embodiments of the invention, the reducing provides dodecane-1,12-diol in at least about a 90 mol.% yield, based upon the lauryl lactone. In more specific embodiments of the invention, the reducing provides dodecane-1,12-diol in at least about a 95 mol.% yield, based upon the lauryl lactone. In more specific embodiments of the invention, the reducing provides dodecane-1,12-diol in at least about a 98 mol.% yield, based upon the lauryl lactone. In more specific embodiments of the invention, the reducing provides dodecane-1,12-diol in at least about a 99 mol.% yield, based upon the lauryl lactone. In more specific embodiments of the invention, the reducing provides dodecane-1,12-diol in about a 98 mol.% yield to about a 99.9 mol.% yield, based upon the lauryl lactone.

In specific embodiments of the invention, the reducing provides one or more by-products that includes at least one of 1-dodecanol, un-reacted starting material, methyl 12-hydroxydodecanoate, 1-undecanol, butane-1,4-diol, and the mono methyl ester of 1,12-dodecanedioic acid. In such embodiments of the invention, the reducing provides by-products that include 1-dodecanol, un-reacted starting material, methyl 12-hydroxydodecanoate, 1-undecanol, butane-1,4-diol, and/or the mono methyl ester of 1,12-dodecanedioic acid. In more specific embodiments of the invention, the reducing provides by-products that include 1-dodecanol, un-reacted starting material, methyl 12-hydroxydodecanoate, 1-undecanol, butane-1,4-diol, and the mono methyl ester of 1,12-dodecanedioic acid.

In specific embodiments of the invention, both the oxidizing and the reducing are carried out without any intermediate workup. In more specific embodiments of the invention, both the oxidizing and the reducing are carried out in the same reaction vessel. In more specific embodiments of the invention, both the oxidizing and the reducing are carried out in the same reaction vessel without any intermediate workup.

### Enumerated Embodiments

Specific enumerated embodiments [1] to [32] provided below are for illustration purposes only, and do not otherwise limit the scope of the disclosed subject matter, as defined by the claims.

The present invention provides a method of producing dodecane-1,12-diol

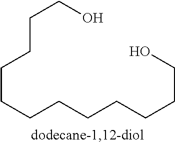 [SMILES: OCCCCCCCCCCCCO]

the method including oxidizing cyclododecanone

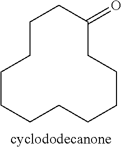 [SMILES: O=C1CCCCCCCCCCC1]

to provide lauryl lactone

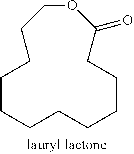 [SMILES: O=C1CCCCCCCCCCCO1]

and reducing the lauryl lactone to the dodecane-1,12-diol.

The present invention also provides a method of embodiment [1], wherein the oxidizing includes (i.e., employs) at least one of hydrogen peroxide, a peracetic acid, a trifluoro peracetic acid, and a peracid.

The present invention also provides a method of embodiment [1], wherein the oxidizing includes (i.e., employs) at least one of hydrogen peroxide, meta-chloroperoxybenzoic acid (mCPBA), trifluoro peracetic acid (CF₃CO₃H), permaleic acid (HO₃CHC═CCO₃H), performic acid (CH₂O₃), peracetic acid (CH₃CO₃H), magnesium monoperoxyphthalate (MMPP), perbenzoic acid (PBA) and monoperphthalic acid (MPPA).

The present invention also provides a method of embodiment [1], wherein the oxidizing includes (i.e., employs) hydrogen peroxide, maleic anhydride, and methyl acetate.

The present invention also provides a method of embodiment [1], wherein the oxidizing includes (i.e., employs) hydrogen peroxide, maleic anhydride, and methyl acetate, which is carried out by the simultaneous addition of the hydrogen peroxide and the maleic anhydride in the methyl acetate, to a solution of the cyclododecanone in the methyl acetate.

The present invention also provides a method of embodiment [1], wherein the oxidizing includes (i.e., employs) hydrogen peroxide, maleic anhydride, and methyl acetate, which is carried out by the addition of the cyclododecanone to a preformed solution of hydrogen peroxide and maleic anhydride.

The present invention also provides a method of embodiment [1], wherein the oxidizing includes (i.e., employs) hydrogen peroxide, maleic anhydride, and methyl acetate, which is carried out by the addition of hydrogen peroxide to a solution of cyclododecanone and maleic anhydride in a solvent.

The present invention also provides a method of any one of embodiments [1]-[7], wherein the oxidizing includes (i.e., employs) hydrogen peroxide, present in about 1.5 molar equivalents to about 2.5 molar equivalents, relative to the cyclododecanone.

The present invention also provides a method of any one of embodiments [1]-[8], wherein the oxidizing includes (i.e., employs) maleic anhydride and hydrogen peroxide, present in a molar ratio of about 0.5 to about 1.5.

The present invention also provides a method of any one of embodiments [1]-[9], wherein the oxidizing is carried out at a temperature of about 45° C. to about 60° C.

The present invention also provides a method of any one of embodiments [1]-[10], wherein the oxidizing is carried out for a period of time of about 15 hours to about 30 hours.

The present invention also provides a method of any one of embodiments [1]-[11], wherein the lauryl lactone is obtained in at least about a 95 mol.% yield, relative to the cyclododecanone.

The present invention also provides a method of any one of embodiments [1]-[12], wherein the lauryl lactone is obtained in at least about a 80 mol.% selectivity, relative to the cyclododecanone.

The present invention also provides a method of any one of embodiments [1]-[13], wherein the oxidizing is carried out in a batch mode, wherein at least about 20 kg of lauryl lactone is obtained, per batch.

The present invention also provides a method of any one of embodiments [1]-[14], wherein maleic acid is produced as a by-product in the oxidizing, and is allowed to precipitate from the reaction mixture.

The present invention also provides a method of any one of embodiments [1]-[15], wherein the oxidizing optionally produces one or more by-products including at least one of 12-hydroxdodecanoic acid, maleic acid, 1,12-dodecanedioic acid, and un-reacted starting material.

The present invention also provides a method of any one of embodiments [1]-[16], wherein the oxidizing is carried out employing a solvent having a boiling point less than about 80° C.

The present invention also provides a method of any one of embodiments [1]-[17], wherein the reducing includes (i.e., employs) hydrogen gas (H₂), solvent, and catalyst, at an elevated pressure and an elevated temperature.

The present invention also provides a method of any one of embodiments [1]-[18], wherein the reducing includes (i.e., employs) hydrogen gas (H₂), catalyst, (C₁-C₆)alkyl substituted with hydroxyl, and water.

The present invention also provides a method of any one of embodiments [1]-[19], wherein the reducing includes (i.e., employs) hydrogen gas (H₂), catalyst, methanol, and water.

The present invention also provides a method of any one of embodiments [1]-[20], wherein the reducing includes (i.e., employs) 2% ruthenium (Ru) on carbon catalyst.

The present invention also provides a method of any one of embodiments [1]-[21], wherein the reducing includes (i.e., employs) 2% ruthenium (Ru) on carbon catalyst containing Re, Sn or a combination thereof.

The present invention also provides a method of any one of embodiments [1]-[22], wherein the reducing is carried out at an elevated pressure of at least about 1000 psig.

The present invention also provides a method of any one of embodiments [1]-[23], wherein the reducing is carried out at an elevated temperature of at least about 100° C.

The present invention also provides a method of any one of embodiments [1]-[24], wherein the reducing is carried out for at least about 12 hours.

The present invention also provides a method of any one of embodiments [1]-[25], wherein the dodecane-1,12-diol is obtained in at least about a 98 mol.% yield, based upon the lauryl lactone.

The present invention also provides a method of any one of embodiments [1]-[26], wherein the reducing optionally produces one or more by-products including at least one of 1-dodecanol, un-reacted starting material, methyl 12-hydroxydodecanoate, 1-undecanol, butane-1,4-diol, and the mono methyl ester of 1,12-dodecanedioic acid.

The present invention also provides a method of any one of embodiments [1]-[27], wherein both the oxidizing and the reducing are carried out without any intermediate workup.

The present invention also provides a method of any one of embodiments [1]-[28], wherein both the oxidizing and the reducing are carried out in the same reaction vessel.

The present invention also provides a method of producing butane-1,4-diol

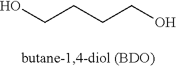

the method including oxidizing cyclododecanone

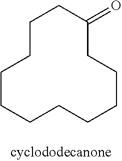 [SMILES: O=C1CCCCCCCCCCC1]

to provide lauryl lactone and maleic acid

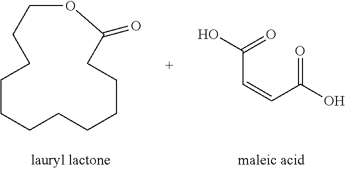 [SMILES: O=C1CCCCCCCCCCCO1 ; O=C(O)/C=C\C(=O)O]

and reducing the maleic acid to butane-1,4-diol.

The present invention also provides a method of producing dodecane-1,12-diol

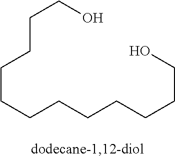 [SMILES: OCCCCCCCCCCCCO]

the method including oxidizing cyclododecanone

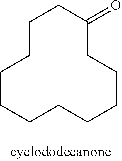 [SMILES: O=C1CCCCCCCCCCC1]

to provide lauryl lactone

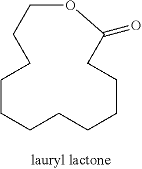 [SMILES: O=C1CCCCCCCCCCCO1]

and reducing the lauryl lactone to the dodecane-1,12-diol,

wherein both the oxidizing and the reducing are carried out without any intermediate workup.

The present invention also provides a method of producing dodecane-1,12-diol

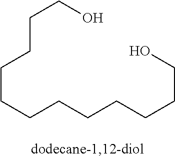 [SMILES: OCCCCCCCCCCCCO]

the method including oxidizing cyclododecanone

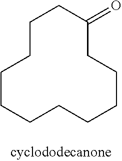 [SMILES: O=C1CCCCCCCCCCC1]

to provide lauryl lactone

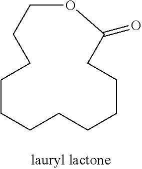 [SMILES: O=C1CCCCCCCCCCCO1]

wherein the oxidizing includes (i.e., employs) hydrogen peroxide, maleic anhydride, and methyl acetate,

wherein the hydrogen peroxide, present in about 1.5 molar equivalents to about 2.5 molar equivalents, relative to the cyclododecanone,

wherein the maleic anhydride and hydrogen peroxide are present in a molar ratio of about 0.5 to about 1.5,

wherein the oxidizing is carried out at a temperature of about 45° C. to about 60° C.,

wherein the oxidizing is carried out for a period of time of about 15 hours to about 30 hours,

wherein the lauryl lactone is obtained in at least about a 95 mol.% yield, relative to the cyclododecanone,

wherein the lauryl lactone is obtained in at least about a 80 mol.% selectivity, relative to the cyclododecanone,

wherein the oxidizing is carried out in a batch mode, wherein at least about 20 kg of lauryl lactone is obtained, per batch,

wherein maleic acid is produced as a by-product in the oxidizing, and is allowed to precipitate from the reaction mixture, and

wherein the oxidizing is carried out employing a solvent having a boiling point less than about 80° C.

and reducing the lauryl lactone to the dodecane-1,12-diol,

wherein the reducing includes (i.e., employs) hydrogen gas (H₂), methanol, water, and 2% ruthenium (Ru) on carbon catalyst containing Re and Sn, at an elevated pressure of at least about 1000 psig, and an elevated temperature of at least about 100° C.,

wherein the reducing is carried out for at least about 12 hours,

wherein the dodecane-1,12-diol is obtained in at least about a 98 mol.% yield, based upon the lauryl lactone, and

wherein both the oxidizing and the reducing are carried out without any intermediate workup.

### EXAMPLES

The present invention can be better understood by reference to the following examples which are offered by way of illustration. The present invention is not limited to the examples given herein.

### Example 1

### Baeyer-Villiger Oxidation of Cyclododecanone (CDDK) Simultaneous Addition, with 2.17 Molar Ratio of H₂O₂/CDDK

A 1-gallon glass jacketed four-necked vessel equipped with a thermocouple, two Claisen heads one containing a 24 in fifty-coiled spiral surface condenser and the other connected to a water deluge reservoir was employed for the Baeyer-Villiger oxidation of cyclododecanone. The vessel was also equipped with a stainless steel mechanical stirrer with a turbine paddle. The fourth opening contains ⅛ in Teflon® tubes for the addition of 70% H₂O₂ in water, and maleic anhydride (MAN)/methyl acetate solutions.

MAN (626.4 g, 6.32 moles) was dissolved in 353 g methyl acetate at room temperature. The mixture was heated for a short time at 50° C. to dissolve all solids. Cyclododecanone (577 g, 3.17 moles) was dissolved in 480 g methyl acetate at 50° C. H₂O₂ (70% in water, 334.3 g, 6.88 moles) was fed into the vessel using an FMI pump. Molar ratios of H₂O₂/ketone and MAN/H₂O₂ were 2.17 and 0.92, respectively. Both the H₂O₂ and the MAN in methyl acetate pumps were started together at flow rates of 2.27 g/min and 11.3 mL/min, respectively. The starting vessel temperature was 46° C.

After complete addition (2 hrs), the temperature rose to 57° C. Subsequently the bath temperature was set at 55-56° C. to maintain temperature at 55° C. After 2 hrs, maleic acid (MA) began to precipitate and was complete after 9 hrs. The product mixture was allowed to cool from 55° C. to 25° C. overnight. Representative sample analysis of the crude product after 22 hr showed 99.1 wt % conversion of CDDK and a molar % selectivity of 94% lauryl lactone based on cyclododecanone. Dodecanedioic acid and 12-hydroxydodecanoic acid were the other co-products produced from cyclododecanone. The mixture was cooled to 25° C. and the solvent methyl acetate removed on a rotary evaporator at 26 in Hg and 50-65° C. Analysis of the methyl acetate solvent showed 4.1 wt % MeOH and a small amount of acetic acid.

Approximately 1965.8 g of crude white maleic acid solid wet with small amounts of LLON was isolated by filtration. Analysis of the crude maleic acid solids showed 1.09 wt. % lauryl lactone, 3.89 wt % dodecandioic acid, 0.944 wt % 12-hydroxydodecanoate and 3.23 wt % mono methyl maleate. The balance was maleic acid.

### Example 1a

### Baeyer-Villiger Oxidation of Cyclododecanone (CDDK) Simultaneous Addition, with 2.1 Molar Ratio of H₂O₂/CDDK

A 1-gallon glass jacketed four-necked vessel equipped with a thermocouple, two Claisen heads one containing a 24 in fifty-coiled spiral surface condenser and the other connected to a water deluge reservoir was employed for the Baeyer-Villiger oxidation of cyclododecanone. The vessel was also equipped with a stainless steel mechanical stirrer with a turbine paddle. The fourth opening contained ⅛ in Teflon® tubes for the addition of 70% H₂O₂ and maleic anhydride (MAN)/methyl acetate solutions. MAN (625.4 g,) was dissolved in 353 g methyl acetate at room temperature. The mixture was heated for a short time at 50° C. to dissolve the solids. Cyclododecanone (577 g, 3.17 moles) was dissolved in 480 g methyl acetate at 50° C. H₂O₂ (70% in water, 324.3) was fed into the vessel using a FMI pump. Molar ratios of H₂O₂/ketone and MAN/H₂O₂ were 2.11 and 0.95, respectively. Both the H₂O₂ and the MAN in methyl acetate pumps were started together at flow rates of 2.27 g/min and 11.3 mL/min, respectively. The starting vessel temperature was 46° C. After complete addition (2 hrs), the temperature rose to 57° C. Subsequently, the bath temperature was raised to 55-56° C. to maintain temperature at 55° C.

After 9 hrs, a precipitate of maleic acid was observed. The product mixture was allowed to cool from 55° C. to 25° C. overnight. Representative sample analysis of the crude product after 22 hr showed 98.5 wt % conversion of CDDK and a molar % selectivity of 90.1% lauryl lactone based on cyclododecanone. Dodecanedioic acid (10.2 mole %) and 12-hydroxydodecanoic acid (1.3 mole %) were the other co-products produced from cyclododecanone. The concentration of H₂O₂ and permaleic acid, measured by Na₂S₂O₃ and Ce(SO₄)₂ titration, remaining in the product was 0.80 wt. % and 8.4 wt. %, respectively.

### Example 2

### Baeyer-Villiger Oxidation of Cyclododecanone (CDDK) Simultaneous Addition

A 500 mL water jacketed 3-necked round-bottomed spherical flask equipped with a thermocouple, Claisen head containing a 12° C. water cooled spiral surface condenser and a 50 mL addition funnel was employed for the Baeyer-Villiger oxidation of cyclododecanone. A circulating water bath was employed for heating and cooling the jacketed vessel. The vessel was also equipped with a mechanical stirrer. Hydrogen peroxide (70% in water, 39.6 g, 0.815 mole) was fed into the vessel using a FMI pump at 3 mL/min employing a ⅛″ Teflon tube inserted down the spiral condenser into the head space of the vessel.

A solution of 98.6 g (0.99 mole) of MAN in 50 mL methyl acetate was charged to the addition funnel at 25° C. CDDK (72.2 g, 0.396 mole) dissolved in 105 mL methyl acetate was charged to the vessel and heated to 45° C. Ten milliliters of the MAN/methyl acetate solution was added first to the ketone/methyl acetate solution. The H₂O₂ pump was started and the MAN/methyl acetate solution in a 50 mL addition funnel added drop wise such that 40 mL of MAN solution was added for every 9 mL 70% H₂O₂. Ice was added to the circulating batch to maintain the temperature at 45° C. Representative sample analysis of the crude product after 22 hr showed 98.2 wt % conversion of CDDK and a molar % selectivity of 81.9% lauryl lactone based on cyclododecanone. Dodecanedioic acid (9.7 molar %) and 12-hydroxydodecanoic acid (1.3 molar %) were the other co-products produced from cyclododecanone. The concentration of H₂O₂ and permaleic acid remaining in the product was 0.04 wt. % and 0.20 wt. %, respectively.

### Example 3

### Baeyer-Villiger Oxidation of Cyclododecanone (CDDK) Addition of H₂O₂ to CDDK and MAN Solution

A 100 mL water jacketed 4-necked round-bottomed spherical flask equipped with a thermocouple, a 12° C. water cooled spiral surface condenser and magnetic stirrer. A circulating water batch was employed for heating and cooling the jacketed vessel. Cyclododecanone (12 g, 0.067 mole) and 17.7 g (0.197 mole) MAN were added to the vessel followed by 17.2 g tetrahydrofuran. The mixture was heated to 46° C. Hydrogen peroxide (70% in water, 6.86 g. 0.141 mole) was added to the vessel in six 2 mL increments. The temperature began to increase, and the exothermic heat was controlled by ice addition to the water bath. The addition occurred over 1 hr. Maleic acid began to precipitate 40 minutes after the addition of H₂O₂. Representative sample analysis of the crude product after 22 hr at 46° C. showed 86.4 wt % conversion of CDDK and a molar % selectivity of 77.8% lauryl lactone based on cyclododecanone. Dodecanedioic acid (10.9 molar %) and 12-hydroxydodecanoic acid (3.8 molar %) were the other co-products produced from cyclododecanone. The concentration of H₂O₂ and permaleic acid remaining in the product was 0.9 wt. % and 1.2 wt. %, respectively.

### Example 4

### Baeyer-Villiger Oxidation of Cyclododecanone (CDDK) Addition of H₂O₂ to CDDK and MAN Solution

A 100 mL water jacketed 4-necked round-bottomed spherical flask equipped with a thermocouple, a 12° C. water cooled spiral surface condenser and magnetic stirrer was employed. A circulating water bath was employed for heating and cooling the jacketed vessel. Cyclododecanone (12.1 g, 0.066 mole) and 14.5 (0.148 mole) MAN were added to the vessel followed by 17.1 g tetrahydropyran. The mixture was heated to 46° C. Hydrogen peroxide (70% in water, 6.81 g. 0.140 mole) was added to the vessel in six 2 mL increments. The temperature began to increase and the exothermic heat was controlled by ice addition to the water bath. The addition required 2 hr to complete. Maleic acid began to precipitate 40 minutes after the addition of H₂O₂. Representative sample analysis of the crude product after 22 hr at 46° C. showed 99.2 wt % conversion of CDDK and a molar selectivity of 84.6 molar % lauryl lactone based on cyclododecanone. Dodecanedioic acid (10.0 molar %) and 12-hydroxydodecanoic acid (2.9 molar %) were the other co-products produced from cyclododecanone. The concentration of H₂O₂ and permaleic acid remaining in the product was 0.13 wt. % and 0.8 wt %, respectively.

### Example 5

### Baeyer-Villiger Oxidation of Cyclododecanone (CDDK) Addition of CDDK to Permaleic Acid

A 100 mL water jacketed 4-necked round-bottomed spherical flask equipped with a thermocouple, a 12° C. water cooled spiral surface condenser and magnetic stirrer was employed. A circulating water bath was employed for heating and cooling the jacketed vessel. MAN (7.53 g, mole) and 14.9 g dimethyl succinate (DBE4) solvent were added to the vessel at 25° C. The solution was heated to 38° C. H₂O₂ (70% in water, 2.52 g, 0.052 mole) was added in three equal increments at 35° C., followed by raising the temperature to 48-49° C. and holding for 1 hr. This maximized the formation of permaleic acid. Solid cyclododecanone (4.57 g, 0.025 mole) was added in three equal increments. No exothermic heat was observed. The solution became turbid due to maleic acid precipitation after 2.5 hr run time. The mixture was heated to 46° C. and run for an additional 20 hr. Representative sample analysis of the crude product after 22 hr at 46° C. showed 99.1 wt % conversion of CDDK and a molar selectivity of 89.0 molar % lauryl lactone based on cyclododecanone. Dodecanedioic acid (10.1 molar %) and 12-hydroxydodecanoic (0.7 molar %) were the other co-products produced from cyclododcanone.

In a similar manner, other solvents or reaction products can be used in the synthesis. These results and solvents screened at in Table 1.

### Example 6

### Baeyer-Villiger Oxidation of Cyclododecanone (CDDK)

A 100 mL water jacketed 4-necked round-bottomed spherical flask equipped with a thermocouple, a 12° C. water cooled spiral surface condenser and magnetic stirrer was employed. A circulating water bath was employed for heating and cooling the jacketed vessel. MAN (5.95, 0.060 mole) and 14.1 g 1,12-dimethyldodecanedioate were added to the vessel at 25° C. The solution was heated to 38° C. H₂O₂ (70% in water, 1.94 g, 0.040 mole) was added in three increments at 35° C., followed by raising the temperature to 48-49° C. and holding for 1 hr. This maximized the formation of permaleic acid. Solid cyclododecanone (3.55 g, 0.019 mole) was added in three increments. No exothermic heat was observed. The solution became turbid due to maleic acid precipitation after 2 hr run time. The mixture was heated to 46° C. and run for an additional 20 hr. Representative sample analysis of the crude product after 22 hr at 46° C. showed 97.5 wt % conversion of CDDK and a molar selectivity of 92.0 molar % lauryl lactone based on cyclododecanone. Dodecanedioic acid (7.2 molar %) and 12-hydroxydodecanoic (1.3 molar %) were the other co-products produced from cyclododecanone.

### Example 7

### Reduction of Crude BV Product in Water and Methanol

A mixture of 64.1 g crude BV product, 22 g methanol, 22 g water and 5 g of 2% Ru on carbon catalyst containing 6% Re and 0.9% Sn were charged to a 300 mL Stainless Steel autoclave containing a thermocouple, cooling coil, baffle and stirrer. The BV product contained 39.4 wt % maleic acid, 29.3 wt % LLON, 1.9 wt % 12-hydroxydodecanoic acid, 3.5 wt % 1,12-dodecanedioic acid, 3.4 wt % 12-methyl dodecane of 1,12-dodecandioic acid, and 12.5 wt % water. The vessel was flushed first with nitrogen followed by hydrogen and was pressurized to 2000 psig with hydrogen. Stirring at 1800 rpm was commenced and the reactor was heated to 195° C. The hydrogenation was run for 24 hr. Analysis of the product showed a combined 99.8 wt % conversion of LLON, DDDA and HDDA with 88.9% molar selectivity to C12LD. Also produced was 1-dodecanol (6.4 molar %) along with un-reacted methyl 12-hydroxydodecanoate.

### Example 8

### Reduction of Crude BV Product in Water and Methanol at 2500 Psig

A mixture of 60 crude BV product, 24 g methanol, 22 g water and 5 g of 2% Ru on carbon catalyst containing 6% Re and 1.4% Sn were charged to a 300 mL Stainless Steel autoclave containing a thermocouple, cooling coil, baffle and stirrer. The BV product contained 39.4 wt % maleic acid, 29.3 wt % LLON, 1.9 wt % 12-hydroxydodecanoic acid, 3.5 wt % 1,12-dodecanedioic acid, 3.4 wt % mono methyl ester of 1,12-dodecandioic acid, and 12.5 wt % water. The vessel was flushed first with nitrogen followed by hydrogen, and pressurized to 2500 psig with hydrogen. Stirring at 1800 rpm was commenced and the reactor was heated to 200° C. The hydrogenation was run for 27 hr. Analysis of the product showed a combined 99.6% conversion of LLON, DDDA and HDDA with 86.7% molar % selectivity to C12LD. Also produced was 1-undecanol (3.5 molar %) along with un-reacted 12-hydroxydodecanoic acid (0.6 molar %) and methyl 12-hydroxydodecanoate (2.4 molar %). The ratio of hydrogenolysis products to C12LD was 0.055.

### Example 9

### Reduction of Crude BV Product in Water and Methanol at 2000 Psig

A mixture of 64.1 crude BV product, 24 g methanol, 32 g water, 22 g water and 5 g of 2% Ru on carbon catalyst containing 6% Re and 0.9% Sn were charged to a 300 mL Stainless Steel autoclave containing a thermocouple, cooling coil, baffle and stirrer. The BV product contained 39.4 wt % maleic acid, 29.3 wt % LLON, 1.9 wt % 12-hydroxydodecanoic acid, 3.5 wt % 1,12-dodecanedioic acid, 3.4 wt % mono methyl ester of 1,12-dodecandioic acid, and 12.5 wt % water. The vessel was flushed first with nitrogen followed by hydrogen, and pressurized to 2000 psig with hydrogen. Stirring at 1800 rpm was commenced and the reactor was heated to 200° C. The hydrogenation was run for 24 hr. Analysis of the product showed a combined 99.8 wt % conversion of LLON, DDDA and HDDA with 88.8% molar selectivity to C12LD. Also produced was 1-undecanol (6.4 molar %) along with 12-hydroxydodecanoic acid (1.2 molar %) and methyl 12-hydroxydodecanoate (4.9 molar %). The ratio of hydrogenolysis products to C12LD was 0.099.

### Example 10

### Reduction of Crude BV Product in Water and Methanol

A mixture of 54.8 g crude BV product, 26 g methanol, 30 g water and 5 g of 2% Ru on carbon catalyst containing 6% Re and 1.2% Sn were charged to a 300 mL Stainless Steel autoclave containing a thermocouple, cooling coil, baffle and stirrer. The BV product contained 39.4 wt % maleic acid, 29.3 wt % LLON, 1.9 wt %, 12-hydroxydodecanoic acid, 3.5 wt % 1,12-dodecanedioic acid, 3.4 wt % methyl 12-hydroxydodecanoate, and 12.5 wt % water. The vessel was flushed first with nitrogen followed by hydrogen, and pressurized to 2000 psig with hydrogen. Stirring at 1800 rpm was commenced and the reactor was heated to 197° C. The hydrogenation was run for 27 hr. Analysis of the product showed a combined 99.9 wt % conversion of LLON, DDDA and HDDA with 78% molar selectivity to C12LD. Also produced was 1-dodecanol (2.8 molar %) along with un-reacted 12-hydroxydodecanoic acid (2.5 molar %) and methyl 12-hydroxydodecanoate (8.6 molar %).

### Example 11

### Reduction of 1,12-Dodecandioic Acid in Water and Methanol with No Re in the Catalyst

A mixture of 20 g (0.087 mole) 1,12-dodecandioic acid, 32 g methanol, 18 g water and 5 g of 5% Ru on carbon catalyst containing 1% Sn are was charged to a 300 mL Stainless Steel autoclave containing a thermocouple, cooling coil, baffle and stirrer. The vessel was flushed first with nitrogen followed by hydrogen, and pressurized to 2500 psig with hydrogen. Stirring at 1800 rpm was commenced and the reactor was heated to 230° C. The hydrogenation was run for 22 hr. Analysis of the product showed a 98.4 wt % conversion of DDDA with 30.7% molar selectivity to C12LD. Also produced was 1-dodecanol (1.8 molar %) along with the intermediate 12-hydroxydodecanoic acid (14.4 molar %) and the mono methyl ester of 1,12-dodecanedioic acid (38 molar %). It can be seen from the low molar yield to C12LD, the un-reacted intermediate 12-hydroxydodecanoic acid and mono methyl ester of 1,2-dodecanedioic acid that the absence of Re had a dramatic effect on the reduction rate to C12LD.

TABLE 1
Addition of CDDK to Permaleic Acid in Different Solvents

| | | | | CDDK | LLON | DDDA | HDDA |
|---|---|---|---|---|---|---|---|
| | H₂O₂/CDDK | MAN/H₂O₂ | MAN/CDDK | conv | % mole | % mole | % mole |
| Solvent | mole ratio | mole ratio | mole ratio | wt % | sel | sel | sel |
| Dioxane | 2.1 | 1.4 | 2.9 | 97.2 | 83.6 | 11.1 | 0.8 |
| Ethyl Propionate | 2.1 | 1.4 | 2.8 | 99.2 | 84.9 | 9.4 | 0.9 |
| Ethyl Acetate | 2.1 | 1.4 | 3.0 | 97.2 | 85.4 | 12.5 | 1.2 |
| Dibasic esters* | 2.1 | 1.5 | 3.0 | 99.1 | 89.0 | 10.1 | 0.7 |
| 1,12- | 2.05 | 1.5 | 3.1 | 97.5 | 92 | 7.2 | 0.1 |
| dimethyldodecanediol | | | | | | | |
| Methyl acetate | 2.1 | 1.34 | 2.8 | 97.4 | 89.2 | 9.6 | 1.2 |
| Ω-pentadecalactone | 2.1 | 1.5 | 3.1 | 99.9 | 6.6 | 6.6 | 0.1 |

Run time is 22 hr, temperature is 46-47° C.,
*dibasic esters are C4 to C6 dimethyl esters of C4 to C6 dibasic acids

Numerous modifications and variations of the presently disclosed subject matter are possible in light of the above teachings. It is therefore to be understood that within the scope of the appended claims, the disclosed subject matter may be practiced otherwise than as specifically described herein.

All publications, patents, and patent applications are incorporated herein by reference. While in the foregoing specification this disclosed subject matter has been described in relation to certain preferred embodiments thereof, and many details have been set forth for purposes of illustration, it will be apparent to those skilled in the art that the disclosed subject matter is susceptible to additional embodiments and that certain of the details described herein may be varied considerably without departing from the basic principles of the disclosed subject matter.

1. A method of producing dodecane-1,12-diol 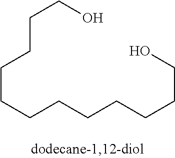 [SMILES: OCCCCCCCCCCCCO]
the method comprising oxidizing cyclododecanone 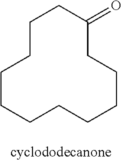 [SMILES: O=C1CCCCCCCCCCC1]
to provide lauryl lactone 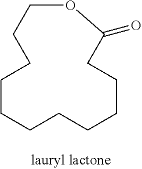 [SMILES: O=C1CCCCCCCCCCCO1]
and reducing the lauryl lactone to the dodecane-1,12-diol, wherein both the oxidizing and the reducing are carried out without any intermediate workup.

2. The method of claim 1, wherein the oxidizing comprises at least one of hydrogen peroxide, a peracetic acid, a trifluoro peracetic acid, and a peracid.

3. The method of claim 1, wherein the oxidizing comprises at least one of hydrogen peroxide, meta-chloroperoxybenzoic acid (mCPBA), trifluoro peracetic acid (CF₃CO₃H), permaleic acid (HO₃CHC═CCO₃H), performic acid (CH₂O₃), peracetic acid (CH₃CO₃H), magnesium monoperoxyphthalate (MMPP), perbenzoic acid (PBA) and monoperphthalic acid (MPPA).

4. The method of claim 1, wherein the oxidizing comprises hydrogen peroxide, maleic anhydride, and methyl acetate.

5. The method of claim 1, wherein the oxidizing comprises hydrogen peroxide, maleic anhydride, and methyl acetate, which is carried out by the simultaneous addition of the hydrogen peroxide and the maleic anhydride in the methyl acetate, to a solution of the cyclododecanone in the methyl acetate.

6. The method of claim 1, wherein the oxidizing comprises hydrogen peroxide, maleic anhydride, and methyl acetate, which is carried out by the addition of the cyclododecanone to a preformed solution of hydrogen peroxide and maleic anhydride.

7. The method of claim 1, wherein the oxidizing comprises hydrogen peroxide, maleic anhydride, and methyl acetate, which is carried out by the addition of hydrogen peroxide to a solution of cyclododecanone and maleic anhydride in a solvent.

8. The method of claim 1, wherein the oxidizing comprises hydrogen peroxide, present in about 1.5 molar equivalents to about 2.5 molar equivalents, relative to the cyclododecanone.

9. The method of claim 1, wherein the oxidizing comprises maleic anhydride and hydrogen peroxide, present in a molar ratio of about 0.5 to about 1.5.

10. The method of claim 1, wherein the oxidizing is carried out at a temperature of about 45° C. to about 60° C.

11. The method of claim 1, wherein the oxidizing is carried out for a period of time of about 15 hours to about 30 hours.

12. The method of claim 1, wherein the lauryl lactone is obtained in at least about a 95 mol.% yield, relative to the cyclododecanone.

13. The method of claim 1, wherein the lauryl lactone is obtained in at least about a 80 mol.% selectivity, relative to the cyclododecanone.

14. The method of claim 1, wherein the oxidizing is carried out in a batch mode, wherein at least about 20 kg of lauryl lactone is obtained, per batch.

15. The method of claim 1, wherein maleic acid is produced as a by-product in the oxidizing, and is allowed to precipitate from the reaction mixture.

16. The method of claim 1, wherein the oxidizing optionally produces one or more by-products comprising at least one of 12-hydroxdodecanoic acid, maleic acid, 1,12-dodecanedioic acid, and un-reacted starting material.

17. The method of claim 1, wherein the oxidizing is carried out employing a solvent having a boiling point less than about 80° C.

18. The method of claim 1, wherein the reducing comprises hydrogen gas (H₂), solvent, and catalyst, at an elevated pressure and an elevated temperature.

19. The method of claim 1, wherein the reducing comprises hydrogen gas (H₂), catalyst, (C₁-C₆)alkyl substituted with hydroxyl, and water.

20. The method of claim 1, wherein the reducing comprises hydrogen gas (H₂), catalyst, methanol, and water.

21. The method of claim 1, wherein the reducing comprises 2% ruthenium (Ru) on carbon catalyst.

22. The method of claim 1, wherein the reducing comprises 2% ruthenium (Ru) on carbon catalyst containing Re, Sn or a combination thereof.

23. The method of claim 1, wherein the reducing is carried out at an elevated pressure of at least about 1000 psig.

24. The method of claim 1, wherein the reducing is carried out at an elevated temperature of at least about 100° C.

25. The method of claim 1, wherein the reducing is carried out for at least about 12 hours.

26. The method of claim 1, wherein the dodecane-1,12-diol is obtained in at least about a 98 mol.% yield, based upon the lauryl lactone.

27. The method of claim 1, wherein the reducing optionally produces one or more by-products comprising at least one of 1-dodecanol, un-reacted starting material, methyl 12-hydroxydodecanoate, 1-undecanol, butane-1,4-diol, and the mono methyl ester of 1,12-dodecanedioic acid.

28. (canceled)

29. The method of claim 1, wherein both the oxidizing and the reducing are carried out in the same reaction vessel.

30. (canceled)

31. (canceled)

32. A method of producing dodecane-1,12-diol 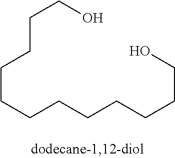 [SMILES: OCCCCCCCCCCCCO]
the method comprising oxidizing cyclododecanone 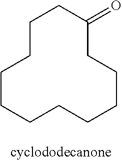 [SMILES: O=C1CCCCCCCCCCC1]
to provide lauryl lactone 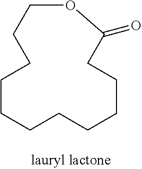 [SMILES: O=C1CCCCCCCCCCCO1]
wherein the oxidizing comprises hydrogen peroxide, maleic anhydride, and methyl acetate,
wherein the hydrogen peroxide, present in about 1.5 molar equivalents to about 2.5 molar equivalents, relative to the cyclododecanone,
wherein the maleic anhydride and hydrogen peroxide are present in a molar ratio of about 0.5 to about 1.5,
wherein the oxidizing is carried out at a temperature of about 45° C. to about 60° C.,
wherein the oxidizing is carried out for a period of time of about 15 hours to about 30 hours,
wherein the lauryl lactone is obtained in at least about a 95 mol.% yield, relative to the cyclododecanone,
wherein the lauryl lactone is obtained in at least about a 80 mol.% selectivity, relative to the cyclododecanone,
wherein the oxidizing is carried out in a batch mode, wherein at least about 20 kg of lauryl lactone is obtained, per batch,
wherein maleic acid is produced as a by-product in the oxidizing, and is allowed to precipitate from the reaction mixture, and
wherein the oxidizing is carried out employing a solvent having a boiling point less than about 80° C.
and reducing the lauryl lactone to the dodecane-1,12-diol,
wherein the reducing comprising hydrogen gas (H₂), methanol, water, and 2% ruthenium (Ru) on carbon catalyst containing Re and Sn, at an elevated pressure of at least about 1000 psig, and an elevated temperature of at least about 100° C.,
wherein the reducing is carried out for at least about 12 hours,
wherein the dodecane-1,12-diol is obtained in at least about a 98 mol.% yield, based upon the lauryl lactone, and
wherein both the oxidizing and the reducing are carried out without any intermediate workup.
